(19) United States
(12) Patent Application Publication
(10) Pub. No.: US 2025/0109372 A1
(43) Pub. Date: Apr. 3, 2025

(54) CELL DETACHMENT APPARATUS

(71) Applicant: CANON KABUSHIKI KAISHA, Tokyo (JP)

(72) Inventors: TAKUJI OKAMOTO, Tokyo (JP); TAKAYUKI TSUKIMOTO, Kanagawa (JP); NORIHIRO SUZUKI, Kanagawa (JP); KEIICHIRO TSUBAKI, Tokyo (JP); TAKAAKI FURUI, Tokyo (JP)

(21) Appl. No.: 18/894,555

(22) Filed: Sep. 24, 2024

(30) Foreign Application Priority Data
Sep. 29, 2023 (JP) 2023-169957

(51) Int. Cl. C12M 1/00; C12M 1/26

(52) U.S. Cl. CPC C12M 47/02; C12M 23/38; C12M 23/50; C12M 33/08

(57) ABSTRACT

There is provided a cell detachment apparatus that separates a cell attached to a culture surface of a culture substrate from the culture surface by applying vibrations to the culture surface that includes a vibration unit including a vibrator that generates the vibrations, and a first holding unit that holds the culture substrate in a horizontal direction so that a position of the culture substrate with respect to the vibrator is allowed to be changed.

### BACKGROUND

### Field

The present invention relates to a cell detachment apparatus.

### Description of the Related Art

For the purpose of treatment and research and development in the field of medicine, a cell or the like may be cultured on a culture plate or the bottom of a container, such as a dish (petri dish), and used. However, an adherent cell is attached to the bottom of the container. For this reason, the adherent cell needs to be separated and taken out from the culture container.

Japanese Patent Laid-Open No. 2008-092857 describes a cell detachment apparatus that includes a separator that separates a desired live cell in a live cell group from a wall surface of a container by application of ultrasonic waves.

Japanese Patent Laid-Open No. 2015-043751 describes a vibrating apparatus that removes air bubbles generated in cultivation by vibrating the culture container.

### SUMMARY OF THE INVENTION

Some inconvenience has been found in the cell detachment apparatus of Japanese Patent Laid-Open No. 2008-092857 and the vibrating apparatus in Japanese Patent Laid-Open No. 2015-043751. In other words, the cell detachment apparatus in Japanese Patent Laid-Open No. 2008-092857 has no configuration to fix the container, so the position of the container fluctuates due to vibrations generated by application of ultrasonic waves, with the result that a cell possibly cannot be separated.

The vibrating apparatus of Japanese Patent Laid-Open No. 2015-043751 describes a configuration to hold the culture container by biasing the culture container in a vertical direction in order to hold the culture container. However, according to the study, an appropriate position for applying vibrations varied with this configuration. In other words, a peak position of an amplitude can vary when vibrations are continuously applied, and a peak position of an amplitude can vary when a vibration mode is changed. As a result, with a configuration to hold the position of the culture container only at a certain specific position, it can be difficult to separate a cell or the survival rate can decrease as a result of damage to cells.

Therefore, the present disclosure provides a cell detachment apparatus that solves the above inconvenience by not only holding the position of a culture container for a cell but also allowing the position to be changed.

An aspect of the present disclosure provides a cell detachment apparatus that separates a cell attached to a culture surface of a culture substrate from the culture surface by applying vibrations to the culture surface. The cell detachment apparatus includes a vibration unit including a vibrator that generates the vibrations, and a first holding unit that holds the culture substrate so that a position of the culture substrate in a horizontal direction with respect to the vibrator is allowed to be changed.

Further features of the present disclosure will become apparent from the following description of exemplary embodiments with reference to the attached drawings.

### BRIEF DESCRIPTION OF THE DRAWINGS

FIG. 1 is a view that shows a cell detachment apparatus according to an embodiment of the present disclosure.

FIGS. 2A to 2C are views that show a cell detachment apparatus according to a first embodiment of the present disclosure.

FIGS. 3A to 3E are views that show a cell detachment apparatus according to a second embodiment of the present disclosure.

FIGS. 4A to 4E are views that show a cell detachment apparatus according to a third embodiment of the present disclosure.

FIG. 5 is a view that shows a cell detachment apparatus according to a fourth embodiment of the present disclosure.

FIGS. 6A to 6C are views that show a cell detachment apparatus according to a fifth embodiment of the present disclosure.

FIGS. 7A to 7D are views that show examples of a weight in a cell detachment apparatus according to a sixth embodiment of the present disclosure.

FIGS. 8A to 8D are views that show a cell detachment apparatus according to a seventh embodiment of the present disclosure.

FIG. 9 is a view that shows a cell detachment apparatus according to an eighth embodiment of the present disclosure.

FIG. 10 is a view that shows a cell detachment apparatus according to a ninth embodiment of the present disclosure.

FIGS. 11A to 11D are views that show a cell detachment apparatus according to a tenth embodiment of the present disclosure.

FIG. 12 is a view that shows a cell detachment apparatus according to an eleventh embodiment of the present disclosure.

### DESCRIPTION OF THE EMBODIMENTS

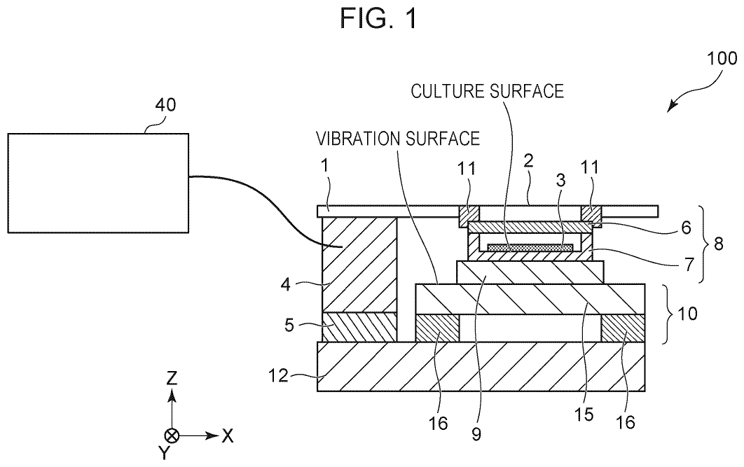

A cell detachment apparatus according to an embodiment of the present disclosure will be described with reference to FIG. 1; however, the present disclosure is not limited thereto. FIG. 1 is a schematic sectional view of the cell detachment apparatus according to the present embodiment.

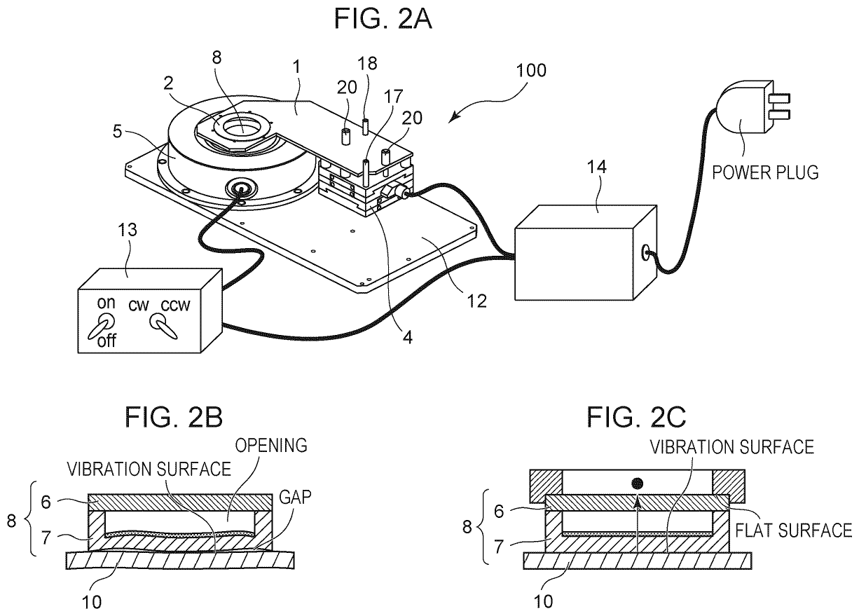

The cell detachment apparatus 100 according to the present embodiment is the cell detachment apparatus 100 that separates a cell 3 attached to a culture surface of a culture substrate 8 from the culture surface by applying vibrations to the culture surface. The cell detachment apparatus 100 includes a vibration unit 10 including a vibrator 16 that generates vibrations, and a first holding unit 1 that holds the culture substrate 8 so that a position of the culture substrate 8 in a horizontal direction with respect to the vibrator 16 is allowed to be changed. In FIG. 1, the horizontal direction is a direction of a plane defined by an X-axis and a Y-axis, and a vertical direction is a Z-axis direction. The same applies to the sectional views of FIG. 2A and subsequent drawings.

Here, the phrase “holds the culture substrate 8 so that a position of the culture substrate 8 in a horizontal direction is allowed to be changed” means that the position of the culture substrate 8 is allowed to be changed in a state where no vibrations are applied and, after the change of the position completes, the culture substrate 8 can be held (fixed) such that the position of the culture substrate 8 does not fluctuate. By generating vibrations with the vibrator 16 in a state where the culture substrate 8 is held, appropriate vibrations are applied to the culture substrate 8 and the cell 3 attached to the culture surface of the culture substrate 8.

The first holding unit 1 in the present embodiment allows the position of the culture substrate 8 in the horizontal direction to be changed, so the culture substrate 8 can be installed at a selected position on or above the vibration unit 10. For example, in a case where a position at which the amplitude of vibrations generated by the vibrator 16 and transmitted to the culture substrate 8 is maximum varies, the culture substrate 8 can be changed by the first holding unit 1 to an appropriate position where a detachment rate and a survival rate of the cell 3 increase.

The cell detachment apparatus 100 according to the present embodiment may include a second holding unit 2 that holds a position of the culture substrate 8 in the vertical direction. The second holding unit 2 may be a weight (loading unit) or a pressure-applying unit that applies pressure such that a distance between the culture substrate 8 and the vibrator 16 is reduced. The weight may be configured to include a plurality of weights. The weight may be held by the first holding unit 1 as shown in FIG. 1 or may be suspended from the first holding unit 1. As shown in FIG. 1, the second holding unit 2 may have a fitting portion 11 for being fitted to the culture substrate 8. Since the second holding unit 2 has the fitting portion 11, it is easy to hold the position of the culture substrate 8. The first holding unit 1 in the present embodiment may include a turning mechanism capable of turning about an axis that coincides with a direction perpendicular to a vibration surface of the vibrator 16 as will be described later. The turning mechanism may include a vibration actuator that rotates a screw.

The cell detachment apparatus 100 according to the present embodiment may include a position control unit 14 that controls the position of the culture substrate 8 in the horizontal direction. The position control unit 14 is connected to a stage 4 allowed to move the position of the culture substrate 8 in the horizontal direction in X-Y directions and is capable of controlling movement. The position control unit 14 may be a configuration that controls turning of the first holding unit 1 with the turning mechanism.

The first holding unit 1 may be configured to be displaceable in the vertical direction. Since the first holding unit 1 is configured to be displaceable in the vertical direction, the first holding unit 1 can be retracted in the Z direction or can be retracted in the Z direction and then retracted in the X or Y direction, so it is easy to install and take out the culture substrate 8.

The culture substrate 8 in the present embodiment may include a container 7 having the culture surface for the cell 3 and an opening, and a lid 6 covering the opening. The first holding unit 1 may be a configuration that holds the lid 6 as shown in FIG. 1 or may be a configuration that holds the container 7.

The cell detachment apparatus 100 according to the present embodiment may have an acoustic transmission medium 9 between the vibration unit 10 and the culture substrate 8. The acoustic transmission medium 9 is provided in a path through which vibrations, particularly, vibrations in an ultrasonic wave band, from the vibration unit 10 to the culture substrate 8 such that a layer of air that causes attenuation of vibrations is not interposed. The acoustic transmission medium 9 may be solid like silicone rubber or may be liquid like water. Alternatively, the acoustic transmission medium 9 may be liquid, and the first holding unit 1 may be configured such that the culture substrate 8 is in contact with the acoustic transmission medium 9 and the culture substrate 8 is not in contact with the vibration unit 10.

The vibration unit 10 in the present embodiment may include a vibration plate 15 for transmitting vibrations generated in the vibrator 16, in addition to the vibrator 16. Vibrations generated by the vibrator 16 may be vibrations in an ultrasonic wave band.

Hereinafter, examples of the cell detachment apparatus 100 according to the embodiment of the present disclosure will be described.

### First Embodiment

An example of a cell detachment apparatus 100 according to a first embodiment of the present disclosure will be described with reference to FIGS. 2A to 2C.

The cell detachment apparatus 100 according to the present embodiment separates a cell 3 from a culture substrate 8 by applying vibrations in an ultrasonic wave band (hereinafter, which may be simply referred to as ultrasonic wave vibrations) to the cell 3 provided on the culture substrate 8 and cultured. The cell detachment apparatus 100 includes a disc-shaped vibration unit 10 configured to include a piezoelectric element (vibrator) 16 secured to a vibration plate 15 (not shown). Electrodes (not shown) for giving an alternating signal are provided on the piezoelectric element 16, and a bottom plate 12 supports the vibration unit 10. Electrodes (not shown) are provided on the piezoelectric element 16 such that the front and back sides have opposite polarities in order to cause stretching vibrations, and a return electrode is also provided on the front side so as to be continuous with the back-side electrode on the front side. By giving an alternating signal to a piezoelectric material, it is possible to excite and apply ultrasonic wave vibrations.

In the cell detachment apparatus 100 according to the present embodiment, ultrasonic wave vibrations are transmitted and applied from the vibration plate 15 of the disc-shaped vibration unit 10 to the culture substrate 8; however, the surfaces of the culture substrate 8 and the vibration plate 15 are not necessarily flat and may have slight warpages as shown in FIG. 2B. In such a case, vibrations can be difficult to be transmitted. To bring the bottom of the container 7 into uniform contact with the vibration plate 15, the weight 2 is loaded on the culture substrate 8 in a good balance by matching the gravity center of the weight (second holding unit) 2 with the center of figure of the culture substrate 8 as shown in FIG. 2C. When there is a gap between the vibration unit 10 and the culture substrate 8, ultrasonic wave vibrations attenuate in the gap, and necessary vibrations can be not obtained, so appropriate loading with the weight 2 is needed. On the other hand, when the weight 2 is too heavy, it can impair vibrations of the vibration unit 10, so loading suitable for the container 7 can be selected.

Here, when ultrasonic wave vibrations are applied to the culture substrate 8, the culture substrate 8 can float by a slight amount of less than or equal to 10 μm together with the weight 2 at an application pressure of ultrasonic wave vibrations, become indeterminate in position, and shift from an initial position. As a manner for determining the position of the culture substrate 8, the vibration unit 10, the weight 2 that is loaded on the culture substrate 8 on the vibration unit 10 in the vertical direction, and the first holding unit 1 that positions the culture substrate 8 and the weight 2 to hold the positions with respect to the vibrator 16. A positioning member 1 in FIG. 2A is an example of the first holding unit 1. With such a configuration, a deviation of a positional relationship between the vibrator 16 and the culture substrate 8 on which a cell is installed from an initial setting can be reduced.

The configuration of the cell detachment apparatus 100 according to the embodiment will be specifically described. Initially, the configuration of the culture substrate 8 including the container 7 will be described. The cell 3 is cultured by the culture substrate 8 configured to include the container 7 and the lid 6 covering the opening of the container 7 and is in a state attached to the bottom of the container 7. The lid 6 provides obstructions so that a contaminant does not touch the cell 3, that is, a contaminant is reduced. The culture substrate 8 is desirably configured such that the lid 6 is fitted to the container 7. With the lid 6, it is possible to reduce contaminants produced as rubbing dust or the like as a result of contact of the weight 2 integrated with the positioning member 1 in a case where opening and placement of the weight 2 are automatically performed, as will be described later.

The vibration unit 10 in the cell detachment apparatus 100 according to the present embodiment has such a shape that the culture substrate 8 can be installed on an upper surface of the vibration unit 10. For example, the bottom of the container 7 is flat, the vibration surface of the vibration unit 10 is similarly flat. A vibration transmission medium for further efficiently transmitting vibrations may be provided between the vibration surface and the culture substrate 8. The vibration transmission medium may be liquid, such as water and glycol, or gel or may be solid, such as silicone rubber. A deformable and easy-to-closely adhere elastic material, such as rubber, can be used as an acoustic transmission member.

As described above, the vibration surface of the vibration unit 10 and the surface of the bottom of the container 7 each are not necessarily flat and may have slight warpage, so vibrations are difficult to be transmitted when there occurs a gap.

For this reason, to bring the bottom of the culture substrate 8 into uniform contact with the vibration surface, loading is performed with a force suitable for warpages of the substrates by using the weight 2. A surface of the weight 2, which contacts with the culture substrate 8, is also desirably flat so that the weight 2 is equally applied to the culture substrate 8 at an uniform pressure.

In the present embodiment, the weight 2 serving as the second holding unit and the positioning member 1 (first holding unit) that holds the weight 2 and that is used to provide instructions so that the position of the container 7 is allowed to be changed. The positioning member 1 may be configured not to constrain an axis in a gravitational direction.

Examples of the positioning member 1 are shown in FIGS. 1 and 2A. In FIG. 1, the positioning member 1 is fixed to the stage 4 movable in the horizontal direction. By intervening the stage 4, a placement position of the culture substrate 8, in which the cell 3 is cultured, on the vibration unit 10 can be set such that a deviation from an appropriate position at the time when vibrations are applied is reduced. Here, an appropriate position is a position at which force is applied to an adhesive interface of the cell 3. An appropriate position can vary with time depending on the progress of detachment and can vary depending on a vibration mode generated by the vibrator 16 or aged degradation of the vibrator 16 itself. The intensity of application appears depending on the shape of the vibration transmission medium 9 or the shape of the vibration plate 15, so it is extremely important to move the culture substrate 8 to an appropriate position at the time when vibrations are applied. For this reason, the position control unit 14 capable of changing the position of the culture substrate 8 by including a stage control circuit that controls the stage 4 and electrically controlling the position of the stage 4 can be provided. A switching unit 13 may be used and configured to switch between an operation to apply vibrations from the vibration unit 10 and an operation to change the position of the culture substrate 8 using the first holding unit 1.

### Second Embodiment

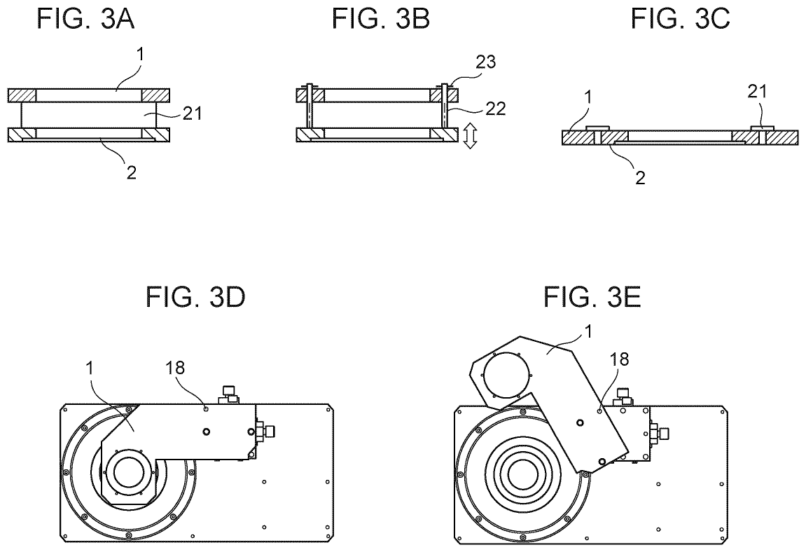

FIGS. 3A to 3E show an example of a cell detachment apparatus according to a second embodiment of the present disclosure.

In a configuration in which a weight 2 (second holding unit) is supported by being fitted to the positioning member 1 (first holding unit), there are a retracting step for the positioning member 1 and an attaching/detaching step for the weight 2. In the configuration according to the present embodiment, even with the weight 2 of which the axis in the gravitational direction is not constrained, the positioning member 1 and the weight 2 are floating in the air and combined as one-piece. FIG. 3A shows a state where the weight 2 is suspended from the positioning member 1 via a suspending member 21, such as a wire (reinforcement fiber, metal wire, music wire, or the like). In FIG. 3B, the weight 2 is suspended with a structure that slide axes 22 are fixed to the weight 2 and fitted to the positioning member 1 and each slide via a ball bearing, a metal bearing, or the like. The weight 2 is movable up and down and can be prevented with retaining rings 23 from falling. The slide axes 22 may be fixed to any one of the positioning member 1 and the weight 2. The suspending member 21 may be made of a soft sheet material or the like, such as rubber (FIG. 3C).

As shown in FIGS. 3D and 3E, the positioning member 1 and the weight 2 can be retracted in one operation, with the result that attaching/detaching of the culture substrate 8 is made easy. FIGS. 3D and 3E respectively show states before and after the positioning member 1 rotates about a rotary mast 18.

When the weight 2 serving as the second holding unit is suspended from the positioning member 1 serving as the first holding unit by a rope or the like, the effect of equalizing loading is obtained. Since loading is caused by gravity, loading is stable, and it is possible to solve the inconvenience that it is difficult to create a stable pressure-equalizing state due to the shape of an elastic member, such as a sandwiching spring, permanent set in fatigue of the elastic member, or the like as described in Japanese Patent Laid-Open No. 2015-043751.

### Third Embodiment

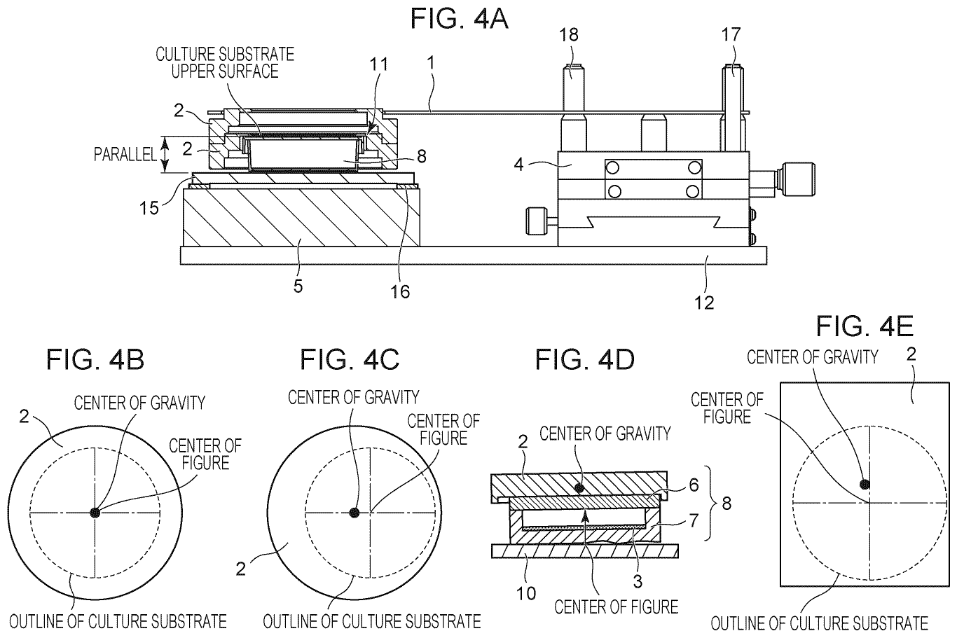

FIGS. 4A to 4E show an example of a cell detachment apparatus according to a third embodiment of the present disclosure.

FIG. 4A is a schematic sectional view of the cell detachment apparatus according to the present embodiment.

A supporting portion 5 and a stage 4 are installed on a bottom plate 12. A vibration unit 10 configured to include a vibration plate 15 and a piezoelectric element 16 (vibrator) is mounted on the supporting portion 5. A culture substrate 8 is placed on an upper surface of the vibration plate 15, and a weight 2 (second holding unit) is disposed parallel in a good balance on a substrate upper surface shown in the drawing so that the culture substrate 8 is difficult to incline. As shown in FIG. 4B, the relationship between the weight 2 and the culture substrate 8 is desirably such that the center of figure of the culture substrate 8 and the position of center of gravity of the weight 2 substantially coincide with each other in the gravitational direction (in a direction normal to the vibration plate 15). FIGS. 4C and 4D show an example in a case of a poor balance and a state where an equalizing pressure is applied and the weight 2 is not parallel to the substrate upper surface. The shape of the weight 2 is not limited to a circular shape and may be another shape. For example, FIG. 4E shows an example in which the weight 2 has a rectangular shape.

In the present embodiment, as shown in FIG. 4A, a deviation can be reduced by providing a fitting portion 11, and a plurality of weights can be selectively disposed to keep a balance.

### Fourth Embodiment

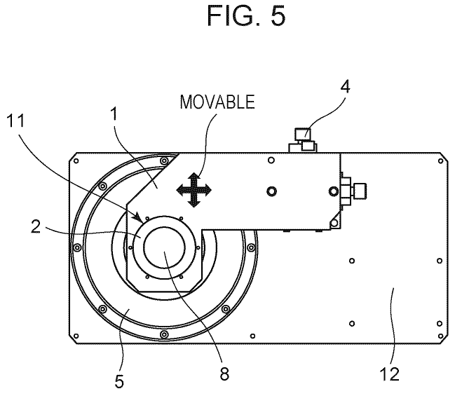

FIG. 5 shows an example of a cell detachment apparatus according to a fourth embodiment of the present disclosure.

FIG. 5 is a top view of the cell detachment apparatus according to the present embodiment. A supporting portion 5 is fixed on a bottom plate 12.

A culture substrate 8 is provided at the center of the supporting portion 5. A weight 2 is placed on the culture substrate 8 and is fitted to a positioning member 1. A stage 4 is provided on the bottom plate 12, and the positioning member 1 is fixed to the stage 4. The positioning member 1 is movable in a direction parallel to a vibration surface of a vibration unit 10 as indicated in the drawing when the stage 4 is moved, and the culture substrate 8 is also movable via the weight 2.

### Fifth Embodiment

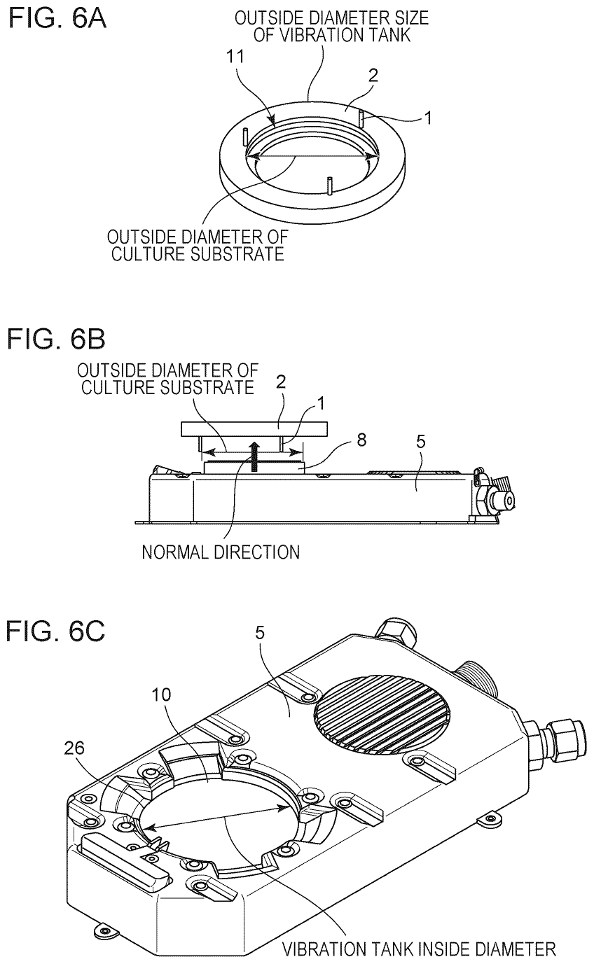

FIGS. 6A to 6C show an example of a cell detachment apparatus according to a fifth embodiment of the present disclosure.

In the present embodiment, a mode in which positioning members 1 are assembled to a weight 2 including a fitting portion 11 for being fitted to a culture substrate 8 is shown.

As shown in FIG. 6A, the positioning members 1 each have a cylindrical pin shape and are equally disposed at three locations. A diameter formed by connecting radially outer peripheries of the cylinders with a circle and an inside diameter of a vibration tank 26 of the cell detachment apparatus shown in FIG. 6C are configured to be fitted to each other. Thus, a similar advantageous effect to that of the second embodiment is obtained. The positioning members 1 and the weight 2 can be retracted in one operation in a normal direction (in the drawing) as shown in FIG. 6B, and attaching/detaching of the culture substrate 8 is made easy.

### Sixth Embodiment

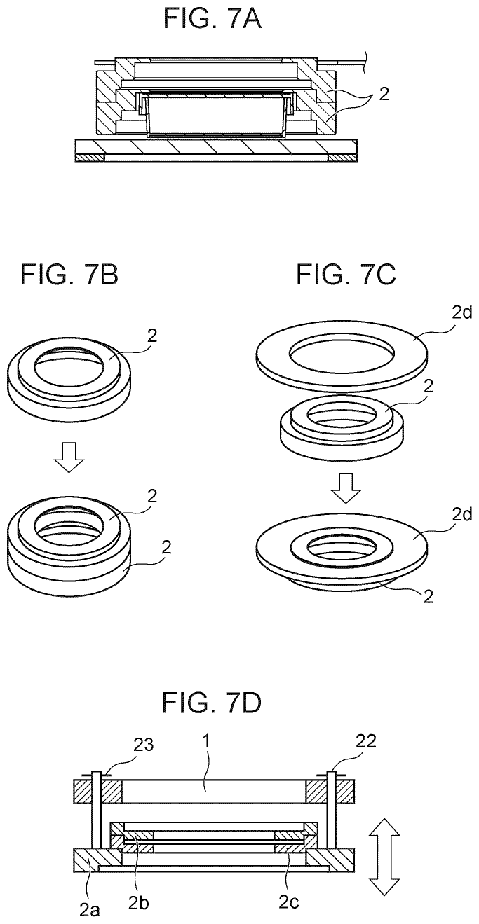

FIGS. 7A to 7D show an example of a cell detachment apparatus according to a sixth embodiment of the present disclosure.

FIG. 7A shows a state when weights 2 are stacked in two stages. FIG. 7B shows a configuration in which weights are stacked in two or more stages. As shown in FIG. 7C, a weight 2 and a weight 2d may have different shapes. When a plurality of the weights 2 is stacked, the stacked weights 2 can be stable as the height is lower, and FIG. 7C can be used in that case.

FIG. 7D shows a mode adapted in the second embodiment. With this configuration, a weight 2a is suspended from a positioning member 1 by slide axes 22, and retaining rings 23 for preventing from falling off are respectively provided at the upper ends of the slide axes 22 to prevent the slide axes 22 from falling off. At this time, a weight 2b and a weight 2c can be stacked on the weight 2a.

### Seventh Embodiment

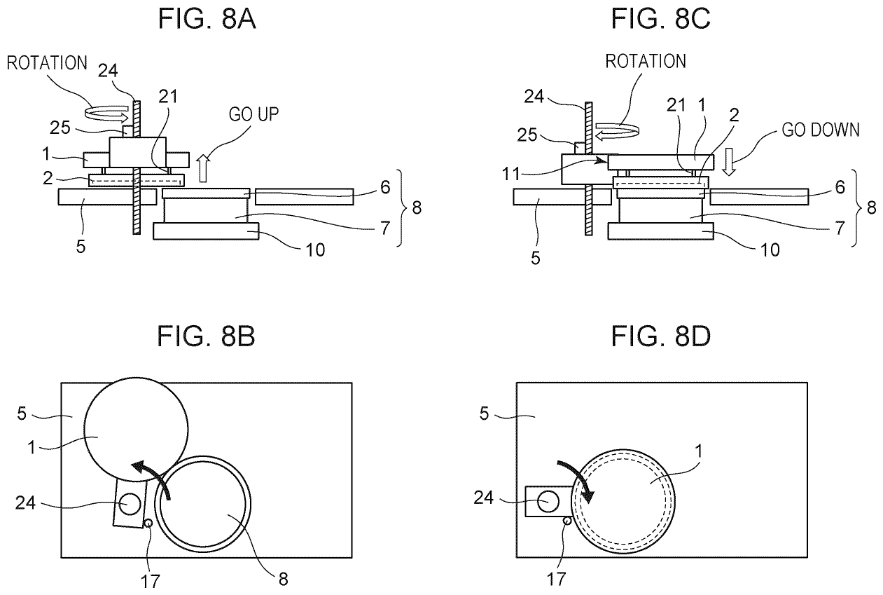

FIGS. 8A to 8D show an example of a cell detachment apparatus according to a seventh embodiment of the present disclosure.

Here, only differences from the first embodiment of the present disclosure will be described.

The cell detachment apparatus according to the present embodiment includes a screw 24 and a friction member 25 on a supporting portion 5, and includes a detent 17. A positioning member 1 is assembled to the screw 24 and co-rotates with rotation of the screw 24. At this time, as the positioning member 1 rotates to the detent 17, the positioning member 1 stops co-rotation, and the positioning member 1 goes up or down as in the case of a general screw.

FIGS. 8A and 8B are a side sectional view and a top view in a state where the positioning member 1 is retracted. At the time of retraction, when a screw rod is rotated in a counterclockwise direction, the positioning member 1 goes up to a position where a lid 6 of a culture substrate 8 is placed. After that, the positioning member 1 leaves from the culture substrate 8, moves to a retracted position, and can be moved up to a selected position. FIGS. 8C and 8D show a state where a weight 2 and the culture substrate 8 are installed so as to be fittable. FIGS. 8C and 8D show a state where, when the screw rod is rotated in a reverse direction, the positioning member 1 reaches a set position, contacts with the detent 17, and goes down. In order for the positioning member 1 to stably rotate at the time of rotation, resistance to a certain extent is desirably applied to a rotation side, and the friction member 25 is a member that applies load to adjust rotational resistance.

For example, if the load is light, the positioning member 1 is pushed back to an opposite side due to an impact caused by striking the detent 17, so such the friction member 25 can be provided.

### Eighth Embodiment

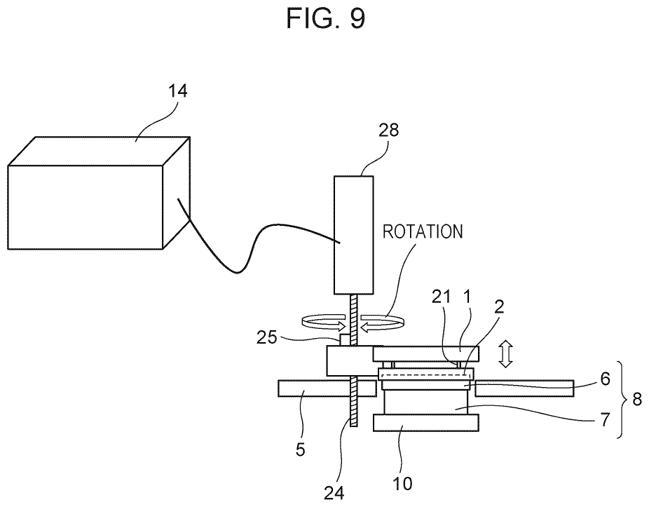

FIG. 9 shows an example of a cell detachment apparatus according to an eighth embodiment of the present disclosure.

Only differences of the cell detachment apparatus according to the present embodiment from the seventh embodiment of the present disclosure will be described.

In the present embodiment, an example in which a rotation actuator 28, which is one of components making up a turning mechanism (a screw 24, a friction member 25, and a rotation actuator 28), is provided and automatic movement of a culture substrate 8 with a first holding unit 1 is shown. As shown in the drawing, the rotation actuator 28 is rotatable in both forward and reverse directions and can be controlled by a position control unit 14 shown in the drawing.

### Ninth Embodiment

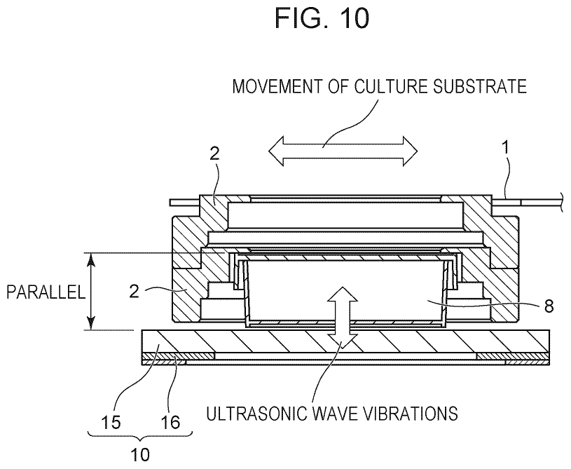

FIG. 10 shows an example of a cell detachment apparatus according to a ninth embodiment of the present disclosure.

In the present embodiment, a vibration unit 10 generates ultrasonic wave vibrations shown in the drawing.

The disc-shaped vibration unit 10 including a piezoelectric element 16 secured to a vibration plate 15 is provided. Electrodes (not shown) for giving an alternating signal are provided on the piezoelectric element 16, and a supporting portion 5 supports the vibration unit 10. Electrodes (not shown) are provided on the piezoelectric element 16 such that the front and back sides have opposite polarities in order to cause stretching vibrations. A return electrode is also provided on the front side so as to be continuous with a back side electrode on the front side. By giving an alternating signal to a piezoelectric material, it is possible to excite and apply ultrasonic wave vibrations. The piezoelectric element 16 does not need to be a ring resonator as shown in FIG. 10, and may be formed of various vibrators with various shapes, such as a Langevin type, a surface acoustic wave type, and a lamination type. When ultrasonic wave vibrations are used, a container is easily movable as shown in the drawing.

### Tenth Embodiment

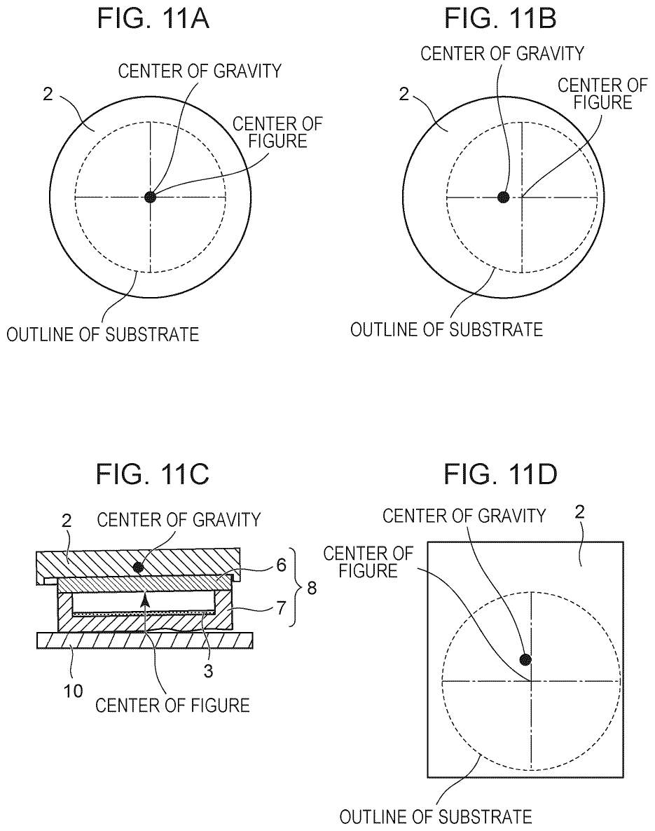

FIGS. 11A to 11D show an example of a cell detachment apparatus according to a tenth embodiment of the present disclosure.

In the cell detachment apparatus according to the present embodiment, a weight 2 has a contact portion with a uniform flat surface as shown in the drawing, and, as shown in FIG. 11A, has a shape such that the weight 2 applies load while the center of gravity of the weight 2 substantially coincides with the center of a projecting upper surface of a culture substrate 8. For further uniform contact with no gap, an advantageous effect can be obtained by interposing a soft elastic material, such as rubber, on the flat surface. FIGS. 11B to 11D show an example of a configuration in which the center of the projecting upper surface of the culture substrate 8 and the center of gravity of the weight 2 do not coincide with each other.

### Eleventh Embodiment

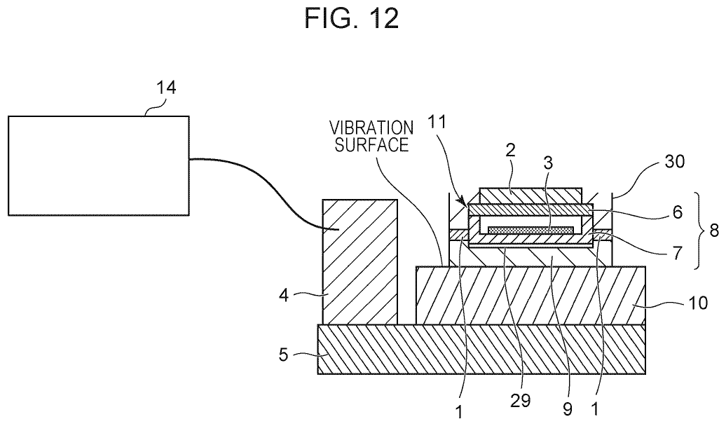

FIG. 12 shows an example of a cell detachment apparatus according to an eleventh embodiment of the present disclosure.

Differences from the configuration of the cell detachment apparatus according to the embodiment of the present disclosure described with reference to FIG. 1 will be described. In the present embodiment, an acoustic transmission medium 9 that is liquid is held by a holding portion 30 for the acoustic transmission medium 9, a culture substrate 8 is in contact with the acoustic transmission medium 9, and the culture substrate 8 is not in contact with a vibration unit 10. A first holding unit 1 holds the culture substrate 8, and a position at which the culture substrate 8 is held can be changed by using, for example, a variable length pin as the first holding unit 1. As shown in FIG. 12, a position of the culture substrate 8 may be configured to be further easily held by a third holding unit 29. When the third holding unit 29 is made of, for example, a mesh material, and the culture substrate 8 is placed on the mesh material and is installed in the acoustic transmission medium 9 that is liquid, it is easy to hold the position of the culture substrate 8 while bringing the acoustic transmission medium 9 into contact with the culture substrate 8.

With the cell detachment apparatus according to the embodiments of the present disclosure, not only the position of the culture container is held, but also the position is allowed to be changed, so the culture container is disposed at an appropriate position and held at that position, with the result that appropriate vibrations are applied. As a result, it is less likely that a cell is difficult to be separated, and the survival rate of the cell is difficult to decrease.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-169957, filed Sep. 29, 2023, which is hereby incorporated by reference herein in its entirety.

What is claimed is:

1. A cell detachment apparatus that separates a cell attached to a culture surface of a culture substrate from the culture surface by applying vibrations to the culture surface, the cell detachment apparatus comprising:
a vibration unit including a vibrator that generates the vibrations; and
a first holding unit that holds the culture substrate so that a position of the culture substrate in a horizontal direction with respect to the vibrator is allowed to be changed.

2. The cell detachment apparatus according to claim 1, further comprising a second holding unit that holds a position of the culture substrate in a vertical direction.

3. The cell detachment apparatus according to claim 2, wherein the second holding unit is a weight.

4. The cell detachment apparatus according to claim 3, wherein the weight is configured to include a plurality of weights.

5. The cell detachment apparatus according to claim 3, wherein the weight is suspended from the first holding unit.

6. The cell detachment apparatus according to claim 2, wherein the second holding unit includes a pressure-applying unit arranged to apply pressure such that a distance between the culture substrate and the vibrator is reduced.

7. The cell detachment apparatus according to claim 1, wherein the first holding unit includes a fitting portion for being fitted to the culture substrate.

8. The cell detachment apparatus according to claim 1, further comprising a turning mechanism capable of turning the first holding unit about an axis that coincides with a direction perpendicular to a vibration surface of the vibrator.

9. The cell detachment apparatus according to claim 8, wherein the turning mechanism includes a vibration actuator that rotates a screw.

10. The cell detachment apparatus according to claim 1, further comprising a position control unit that controls a position of the culture substrate in the horizontal direction with respect to the vibrator.

11. The cell detachment apparatus according to claim 10, further comprising a turning mechanism capable of turning the first holding unit about an axis that coincides with a direction perpendicular to a vibration surface of the vibrator, wherein the position control unit controls turning of the first holding unit with the turning mechanism.

12. The cell detachment apparatus according to claim 1, wherein the first holding unit is configured to be displaceable in a vertical direction.

13. The cell detachment apparatus according to claim 1, wherein the culture substrate includes a container having a culture surface for the cell and an opening, and a lid covering the opening.

14. The cell detachment apparatus according to claim 13, wherein the first holding unit holds the lid.

15. The cell detachment apparatus according to claim 13, wherein the first holding unit holds the container.

16. The cell detachment apparatus according to claim 1, further comprising an acoustic transmission medium between the vibration unit and the culture substrate.

17. The cell detachment apparatus according to claim 16, wherein
the acoustic transmission medium is liquid, and
the first holding unit is configured such that the culture substrate is in contact with the acoustic transmission medium and the culture substrate is not in contact with the vibration unit.

18. The cell detachment apparatus according to claim 1, wherein the vibrator generates vibrations in an ultrasonic wave band.
